(19) United States
(12) Patent Application Publication
(10) Pub. No.: US 2017/0000729 A1
(43) Pub. Date: Jan. 5, 2017

(54) METHODS AND COMPOSITIONS FOR TREATMENT OF NEURODEGENERATIVE DISEASES

(71) Applicant: THE CURATORS OF THE UNIVERSITY OF MISSOURI, Columbia, MO (US)

(72) Inventors: Martin L. Katz, Columbia, MO (US); Joan R. Coates, Columbia, MO (US); Christopher J. Tracy, Columbia, MO (US); Rebecca E.H. Whiting, Columbia, MO (US); Jacqueline W. Pearce, Columbia, MO (US)

(73) Assignee: THE CURATORS OF THE UNIVERSITY OF MISSOURI, Columbia, MO (US)

(21) Appl. No.: 15/125,542

(22) PCT Filed: Mar. 11, 2015
(86) PCT No.: PCT/US2015/019887

§ 371 Date: Sep. 12, 2016

Related U.S. Application Data
(60) Provisional application No. 61/953,658, filed on Mar. 14, 2014.

(51) Int. Cl. A61K 9/00; A61K 9/50; A61K 35/28; A61K 38/48

(52) U.S. Cl. CPC A61K 9/0048; A61K 9/0085; A61K 38/4813; A61K 9/5068; A61K 35/28; C12Y 304/14009

(57) ABSTRACT

The present invention provides methods and compositions for treating a neurodegeneration disease or retinal degenerative disease in a mammal comprising the use of mesenchymal stem cells expressing a therapeutic compound. The invention also provides cells and constructs for use in such methods. Also provided are kits for treatment of a neurodegeneration disease or retinal degenerative disease. The present invention relates in general to the field of retinal degenerative and neurodegenerative diseases. More specifically, the invention relates to methods for treatment of retinal degenerative and neurodegenerative disease.

### CROSS REFERENCE TO RELATED APPLICATIONS

This application claims the benefit of U.S. Provisional Application No. 61/953,658, filed Mar. 14, 2014, herein incorporated by reference in its entirety.

### STATEMENT REGARDING FEDERALLY SPONSORED RESEARCH

This invention was made with partial government support under grant number R01EY023968 awarded by the National Institutes of Health/The National Eye Institute. The government has certain rights in the invention.

### FIELD OF THE INVENTION

The present invention relates in general to the field of retinal degenerative and neurodegenerative diseases. More specifically, the invention relates to methods for treatment of retinal degenerative and neurodegenerative disease.

### BACKGROUND OF THE INVENTION

Age-related macular degeneration and diabetic retinopathy are common retinal degenerative disorders that result in significant visual impairment and even functional blindness in humans. A number of less common inherited diseases also lead to blindness as a result of retinal degeneration. Alzheimer's disease, Parkinson's disease, and amyotrophic lateral sclerosis are among many diseases that involve degeneration of regions of the central nervous system. Some inherited diseases, including many of the lysosomal storage disorders, involve degeneration of both the retina and the central nervous system. For most of these disorders, there is no effective treatment available. Treatments for some of these disorders are under investigation, however the proposed treatments are expensive and involve significant risks and complications. There is a great need for better approaches for treatment of all neurodegenerative diseases.

### SUMMARY OF THE INVENTION

In one aspect, the invention provides a method of treatment or prevention of a neurodegenerative disease or retinal degenerative disease comprising delivery of at least one cell to a subject, wherein said cell provides a therapeutic compound to said subject in an effective amount to reduce disease symptoms. In one embodiment, said cell comprises a construct expressing said therapeutic compound. In another embodiment, delivery of the cell comprises injection into cerebrospinal fluid or the eye. In further embodiments, injection into the cerebrospinal fluid comprises an intracerebroventricular, cisterna magna, or intrathecal injection, or a combination thereof, or injection into the eye comprises injection into the vitreous. In another embodiment, delivery of said at least one cell comprises injection into the cerebrospinal fluid and injection into the eye. In other embodiments, said neurodegenerative disease is a central nervous system degenerative disease or a retinal degenerative disease, or said retinal degenerative disease is a lysosomal storage disease or a neuronal ceroid lipofuscinosis disease. In still further embodiments, said neurodegenerative disease is caused by a mutation in the TPP1 gene, or is characterized by progressive neurodegeneration, accumulation of autofluorescent lysosomal storage bodies, or blindness, or is CLN2-type late-infantile neuronal ceroid lipofuscinosis or a disease homologous to late-infantile neuronal ceroid lipofuscinosis. In other embodiments, said cell is an autologous cell, such as a mesenchymal stem cell. In a further embodiment, said mesenchymal stem cell is isolated from bone marrow, adipose tissue, muscle tissue, blood, or dental pulp.

In a further embodiment, a method of the invention comprises delivery of a population of cells that provides a therapeutic compound to said subject in an effective amount to reduce disease symptoms. In another embodiment, said therapeutic compound comprises a protein, a peptide, a polypeptide, an RNA molecule, a carbohydrate, an antibody or antibody fragment, or a small molecule that is not functionally present in cells of the subject at all or at levels sufficient to prevent disease symptoms. In a specific embodiment, said therapeutic compound is a functional copy of a TPP1 protein, such as a human TPP1 protein or a recombinant TPP1 protein. In another embodiment, the subject is selected from the group consisting of murine, canine, feline, and human.

In another aspect, the invention provides a construct for treating a neurodegenerative disease in a subject, the construct comprising a sequence expressing a therapeutic protein or peptide operably linked to a promoter that functions in an animal cell, wherein delivery of the construct to the subject in an effective amount reduces disease symptoms in the subject. In one embodiment, the invention provides a cell comprising such a construct.

In another aspect, the invention provides a composition for treating a neurodegenerative disease in a subject comprising at least one cell expressing a therapeutic protein or peptide in an effective amount to reduce disease symptoms and a carrier. In one embodiment, the cell is an autologous cell, such as a mesenchymal stem cell. In another embodiment, the mesenchymal stem cell is isolated from bone marrow, adipose tissue, muscle tissue, blood, or dental pulp. In further embodiments, the cell is delivered to the subject by injection into the cerebrospinal fluid or the eye, or the cell is delivered to the subject by injection into the cerebrospinal fluid and injection into the eye. In other embodiments, injection into the cerebrospinal fluid comprises injection into the subarachnoid space or a ventricular space, or injection into the eye comprises injection into the vitreous. In another embodiment, the subject is selected from the group consisting of murine, canine, feline, and human.

In another aspect, the invention provides a kit for treating a neurodegenerative disease in a subject comprising at least one cell from the subject expressing a therapeutic compound for treating a neurodegenerative disease in the subject.

### BRIEF DESCRIPTION OF THE DRAWINGS

For a more complete understanding of the features and advantages of the present invention, reference is now made to the detailed description of the invention along with the accompanying figures and in which:

FIG. 1—(A) shows that pro-TPP1 binds to mannose-6-phosphate receptors on the cell surface. The bound protein is taken up into cells via invagination of the cell membrane to form clathrin-coated pits. These pits are pinched off from the plasma membrane and form vesicles via which the pro-enzyme is delivered to lysosomes. In the lysosome, the protein is released from the membrane-bound receptor and is activated by proteolytic cleavage to a mature form. (B) shows that proteins such as TPP1, when infused into the cerebrospinal fluid, are distributed widely to most brain regions and the optic nerve via the CSF circulation.

FIG. 2—Shows that the retina of a 10.5 month old Dachshund homozygous for the mutant TPP1 allele (A) is much thinner than that of an age-matched homozygous normal Dachshund (B), primarily as a result of thinning of the inner retinal layers. Autofluorescent inclusions are present in the retina of a dog that was homozygous for the mutant TPP1 allele (arrows in C). No such inclusions were present in the retinas of normal dogs.

FIG. 3—Shows scanning laser ophthalmoscopy (SLO) examination of the retinas of dogs homozygous for the TPP1 null mutation revealing numerous unusual lesions (arrows in A). These lesions were not present when the dogs were young but developed and became larger and more numerous as the disease progressed. The example shown in (A) is from a dog at end-stage disease. Optical computed tomography (OCT) examination of these lesions demonstrated that they are localized retinal detachments (B and C). The horizontal lines in (B) show the locations of laser scans used to create cross-sectional OCT images of the retina. The scan lines are superimposed on an SLO image of an area of the retina. The darker line shows the location of the scan that was used to create the retinal cross-sectional image in (C). The arrows in (B) point to 3 of the retinal lesions traversed by the line scan used to create the image in (C). The colored arrows in (C) point to regions that correspond to locations shown by the arrows. Comparison of the images in (B) and (C) demonstrate that the lesions seen on SLO correspond to localized retinal detachments.

FIG. 4—Shows fluorescence micrographs of the interior of a mouse eye showing a sheet of eGFP-expressing mouse MSCs in the vitreous of PPT1 knockout mice 20 weeks after implantation. (A) A sheet of donor cells photographed inside the eye after enucleation of the eye, and after removal of the cornea, lens, and iris. (B) After fixation, a cross-section of a similar sheet of implanted donor cells forming a mesh-like network in the vitreous from a cryostat section of the eye.

FIG. 5—Shows a design of the DNA constructs used with an adeno-associated virus, serotype 2, (rAAV2) vector to transduce TPP1−/− canine MCSs for expression of GFP or pro-TPP1. TR: AAV2 terminal repeats; GFP: green fluorescent protein cDNA; TPP1: human pro-TPP1 cDNA; CAG: promoter consisting of (C) the cytomegalovirus (CMV) early enhancer element, (A) the promoter (the first exon and the first intron of chicken beta-actin gene), (G) the splice acceptor of the rabbit beta-globin gene; and Poly-adeninosine monophosphate stretch. Vector constructs were obtained from SignaGen Laboratories (Rockville, Md.).

FIG. 6—Shows fluorescence (A), phase contrast (B), and merged micrographs (C) of rAAV2-GFP transduced cMSCs in vitro cultured from aspirated canine femur bone marrow cells. Almost every cell expresses GFP, and GFP expression was maintained for at least 8 weeks in culture. Autofluorescence (D) and OCT (E) imaging of rAAV2-GFP transduced autologous cMSCs injected into the vitreous of a dog in vivo (color added in Photoshop). Thick arrow: shadow created by cell clump; thin arrow, individual MSCs.

FIG. 7—Illustrates the principle that underlies this invention with a specific example. Shows a diagram illustrating the mechanism by which autologous MSCs implanted into vitreous (donor cells) can supply active TPP1 enzyme to the retina (recipient cells). Both pro-protein and mature enzyme can be exported from the donor cell into the vitreous via the endosomal system. The protein can then bind to mannose-6-phosphate receptors on retinal cells and be taken up and incorporated into the lysosomes of these cells. This mechanism of production of a therapeutic substance by an implanted cell to patients cells can be generalized to all compounds that the donor cell can be engineered to produce.

FIG. 8—Shows computer-assisted counting of axons in optic nerve cross-sections. A segment of optic nerve was fixed and embedded in an epoxy resin. Cross-sections of the retina were cut at a thickness of 1 μm and the sections were stained with toluidine blue. Micrographs of the entire optic nerve section were obtained at a magnification of 400× and stitched together into a single image using Leica software in conjunction with a microscope equipped with an automated stage. The image was then manipulated with Photoshop software to isolate the axons, which stained more lightly than the rest of nerve. The image of the axons was then superimposed on the original image to confirm that all of the axons were recognized and that objects that are not axons were not highlighted. Any errors in the selective recognition of the axons were corrected using Photoshop tools. Finally, the total number of objects recognized as axons was counted automatically using MetaMorph software. The image represents a small area of one of the composite images of an optic nerve cross section.

FIG. 9—Shows representative ERG tracings from a treated dog at 6 and 7 months of age. The left eye was treated while the right eye received non-therapeutic cells to serve as a control. Recordings represent mixed rod and cone responses from a dark-adapted dog to flashes of 10 mcd·s/m².

FIG. 10—Shows a comparison of retinal lesion prevalence in the treated and untreated eye in a single dog, 16 weeks (top) and 23 weeks (bottom) after intravitreal injection of genetically modified stem cells.

FIG. 11—Shows that a single administration of AAV2-CAG-TPP1 to TPP1−/− dogs at 3 months of age preserves cognitive function as measured by T-maze performance. All 3 dogs listed were TPP1−/−. Porthos received a sham treatment and Duchess and McCartney received the TPP1 gene therapy vector.

FIG. 12—Shows a single intraventricular injection of AAV2-CAG-TPP1 significantly prolongs the life span of TPP1−/− dogs. Untreated dogs survive an average of 44 weeks. Dogs treated with intra-CSF TPP1 enzyme replacement therapy live and average of 63 weeks (Katz et al., J Neurosci Res 92:1591-1598, 2014). Dogs treated with AAV2-CAG-TPP1 survive an average of over 72 weeks.

FIG. 13—Shows a light micrograph of a section of the retina from a TPP1−/− dog that was treated with an intraventricular injection of AAV2-CAG-TPP1 at 3 months of age. The dog reached end-stage disease and was euthanized and the eye fixed for histological examination at 33 months of age. Almost all layers of the retina had disappeared by this age.

FIG. 14—Shows a singe injection of TPP1-expressing autologous MSCs preserves cognitive function in a TPP1−/− Dachshund. Cognitive function was assessed at monthly intervals using a T-maze reversal learning test in untreated TPP1−/− Dachshunds (red plot), in untreated TPP1+/+ Dachshunds (black plot), and in a TPP1−/− Dachshund that had been given an intraventricular injection of TPP1 expressing MSCs at 3 months of age (green plot). At every time point assessed to date the performance of the treated affected dog was almost identical to that or normal dogs and much better than that of untreated affected dogs

### DETAILED DESCRIPTION OF THE INVENTION

The invention provides a therapeutic approach for treating neurodegenerative diseases and retinal degenerative diseases in mammals. In one aspect, the present invention provides a method of treatment of a neurodegenerative or retinal degenerative disease comprising delivery of cells expressing a therapeutic agent, such as a protein or peptide to a subject, for instance through delivery to the cerebrospinal fluid (CSF) or the eye, or both. These cells may remain in the area of administration and produce a therapeutic agent, which can then be secreted or released by the cell and provided to surrounding native cells of the subject. The therapeutic agent may be subsequently activated and utilized by the native cells of the subject, thereby preventing or treating a disease that causes degeneration of the central nervous system (CNS) or retina. Methods of the present invention, therefore, provide continuous delivery of a therapeutic agent or compound (e.g., a therapeutic polypeptide) to a subject over long periods of time after only a single treatment. The methods described herein thus provide an advantage over the currently available treatment methods, which require repeated injections of therapeutic agents.

In one embodiment of the invention, cells injected into the eye may be injected into the vitreous of the eye. In another embodiment, cells injected into the CSF may be via intracerebroventricular, cisterna magna, or intrathecal injections, or any method of entry by which cells may be introduced into the CSF. Combined treatment of both the brain and the eye may protect neurons in the entire visual pathway, while ameliorating other neurologic signs associated with the disease. Thus, in a further embodiment, methods of the invention may comprise both injection into the CSF and injection into the eye for treatment of neurodegenerative diseases.

Any therapeutic molecule that may be used to treat a neurodegenerative disease, including, but not limited to, a protein, a peptide, an RNA molecule, an antibody or antibody fragment, a carbohydrate, a small molecule, and the like, is within the scope of the present invention. In some embodiments, treatment methods of the present invention involve direct delivery of a functional copy of a therapeutic molecule or therapeutic compound, such as a protein, that is not functionally present or is deficient in cells of the subject, such as TPP1. In other embodiments, treatment methods of the present invention may involve direct delivery of a vector expressing a functional copy of a protein that is not functionally present in cells of the subject, such as a vector expressing TPP1. In further embodiments, treatment may comprise any combination of delivery of cells, direct delivery of a therapeutic compound or protein, or delivery of a vector expressing a therapeutic compound or protein.

In certain embodiments, methods of the present invention may be used to treat or prevent any neurodegenerative disease, such as a CNS degenerative or retinal degenerative disease or diseases that involve degeneration of both the CNS and retina. Such diseases may include but are not limited to amyotrophic lateral sclerosis (ALS), Parkinson's disease, neuronal ceroid lipofuscinoses (NCLs), age-related macular degeneration, and diabetic retinopathy.

In one embodiment, the present invention provides a treatment method for NCLs, which are inherited, fatal neurodegenerative disorders characterized by progressive neurological symptoms, including seizures, blindness, and cognitive and motor deterioration, eventually causing death. In humans, symptom onset almost always occurs in childhood, usually between the ages of 6 months and 7 years, depending on the disease subtype. Pathologically, NCLs exhibit accumulation of autofluorescent lysosomal storage material throughout the central nervous system accompanied by widespread neuronal death. No effective treatment has yet been developed for any of the NCLs. As described in detail in the Examples, one embodiment of the present invention provides a therapeutic treatment for CLN2. CLN2 is a recessive disorder resulting from mutations in the TPP1 gene, which encodes a soluble lysosomal enzyme that is exchanged between cells via the cellular endocytic pathway. However, one of skill in the art will understand in light of the present disclosure that the methods described herein may be used for other neurodegenerative diseases, as well. Additionally, other genes that may be useful for treatment of neurodegenerative diseases or retinal degenerative diseases, for example neurotrophic factors, antiangiogenic molecules, or the like, are within the scope of the present invention and thus any genes that direct synthesis and release of any potentially therapeutic compounds or homologs of such genes may be used in accordance with the invention.

Soluble lysosomal enzymes that are secreted by cells, such as TPP1, are synthesized as pro-enzymes on the rough endoplasmic reticulum (ER) and are glycosylated as they traverse the ER and Golgi apparatus. These enzymes are then targeted for delivery to lysosomes via terminal mannose-6-phosphate residues in the sugar adducts. Upon reaching the acidic lumen of the lysosome, the inactive pro-enzyme is cleaved to the active form. The pro-TPP1 can reach the lysosomes not only via delivery from the rough ER by way of the Gogi complex, but also if supplied at the cell surface. Most cells, including neurons, express membrane-bound mannose-6-phosphate receptors on the external side of the cell membrane. Mannose-6-phosphate tagged proteins such as pro-TPP1 will bind to these receptors and can then be delivered to the lysosomes via the endosomal system. Once they reach the lysosomes, the pro-proteins are activated just as if they had been delivered after endogenous synthesis. The mature TPP1 retains its mannose-6-phosphate tag, so the mature protein can also reach the lysosomes when supplied exogenously.

In one embodiment, the invention thus provides a method of treating a subject with a neurodegenerative or a retinal degenerative disease, such as a disease comprising lack of expression of a functional TPP1 gene, the method comprising administering or delivering into the patient a therapeutic compound, for example a protein or peptide such as a functional TPP1 protein, through injection of cells engineered to produce and release the functional protein. In embodiments, such a therapeutic compound may be a TPP1 protein, such as a human TPP1 protein or a recombinant TPP1 protein.

In accordance with the invention, cells that may be useful for injection into a subject and expression and release or secretion of a therapeutic compound may be any cell type capable of proliferation in culture and/or capable of expressing or being engineered or induced to produce a therapeutic compound. In certain embodiments, such cells may be heterologous cells isolated from an appropriate source and expanded in culture, or may be autologous cells isolated from the subject and expanded in culture. In another embodiment, such cells may be isolated from the bone marrow of, for example, the humerus, femur, or other bones from which bone marrow may be extracted. Such cells may, in additional embodiments, be isolated from any other tissue harboring mesenchymal stem cells, such as adipose tissue, muscle tissue, blood, or dental pulp. Once extracted, the cells may be cultured in vitro to isolate a stem cell population therein, particularly a mesenchymal stem cell population. Such cells may then be expanded in culture to obtain a particular concentration of stem cells for use with the methods of the invention. Any concentration of stem cells may be used in accordance with the invention that produces the desired therapeutic effect.

In an embodiment, a nucleotide sequence encoding a therapeutic compound, for example a TPP1 protein, may be inserted or added into the cultured cells in such a way that the cells will stably produce mRNA using the introduced nucleotide sequence as a template, and the mRNA will be translated within the cells. For example, in accordance with the invention, an adeno-associated viral vector may be used to insert or transduce a nucleotide sequence encoding a functional TPP1 protein into a stem cell population as described herein. Other vectors or methods such as electroporation may be used to insert such a nucleotide sequence into the cultured cells to produce the desired recombinant cells.

In other embodiments, any type of cell may be used in accordance with the invention, including, but not limited to, differentiated cells, undifferentiated cells, stem cells, including mesenchymal stem cells (MSCs), hematopoietic stem cells (HSCs), embryonic stem cells (ESCs), or the like. One of skill will understand how to isolate a desired stem cell type in light of the present disclosure. In another embodiment, a particular cell type may be isolated and cultured for use in accordance with the invention. The particular cell type may then be cultured in such a way as to induce the cultured cells to differentiate or dedifferentiate into a different cell type. Persons of skill will be able to identify appropriate culture conditions for such techniques.

Accordingly, cells of the invention may be delivered to the subject, for instance to the retinal tissue or the CSF through a variety of routes. In general, any administration route that places the cells in contact with the desired administration location within the subject, such as retinal tissue or into the vitreous of the eye or CSF for delivery to the brain and the rest of the CNS, may be used. In one embodiment of the invention, the cells may be injected intraocularly or directly into the CSF. Suitable intraocular injection routes, include, but are not limited to, retrobulbarly, subconjuctivally, intravitreally, suprachoroidally, and subretinally. These routes may be used singularly, or in combination to achieve the desired effect. In accordance with the invention, injection into the CSF may be via intracerebroventricular, cisterna magna, or intrathecal injections.

Cells for use in the present invention may be derived from a variety of sources. In one embodiment, mesenchymal stem cells may be derived from embryonic mesoderm tissue. In another embodiment, cells may be derived from adult tissues, including, but not limited to bone marrow, peripheral blood, and adipose tissue. It is also within the scope of the invention to isolate multipotent mesenchymal stem cells from tissues such as umbilical cord blood and placenta.

Cells in accordance with the invention may also be derived from a variety of tissues. As noted above, mesenchymal stem cells may be isolated from embryonic tissues, fetal tissues, neonatal tissues, adult tissues, and combinations thereof. It is also within the scope of the invention to derive mesenchymal cells from at least one of fetal cord blood and placenta. The specific tissues that provide a sufficient source of adult mesenchymal stem cells includes, but is not limited to bone marrow, blood, muscle, skin, and adipose tissue.

Cells for treating neurodegenerative disorders and other retinal or neural dysfunctions may be derived from human and non-human sources. For example, in accordance with the invention, cells may be isolated from a canine, a murine, a feline, or a human, among others. Thus, the mesenchymal cells of the invention may be syngeneic, allogeneic, or xenogeneic in nature.

Cells for practicing the invention may be isolated using any suitable technique that produces viable cells capable of performing the functions and methods set out in the present disclosure. The isolation and culture of mesenchymal stem cells is known in the art (see e.g. Werb et al. (1974) J. Biochem. 137:373-385), as are methods for isolating mesenchymal stem cells. Examples of these methods include, but are not limited to, the following, incorporated herein by reference: U.S. Pat. No. 5,486,359; U.S. Pat. No. 6,039,760; U.S. Pat. No. 6,471,958; U.S. Pat. No. 5,197,985; U.S. Pat. No. 5,226,914; WO92/22584; U.S. Pat. No. 5,827,735; U.S. Pat. No. 5,811,094; U.S. Pat. No. 5,736,396; U.S. Pat. No. 5,837,539; U.S. Pat. No. 5,837,670; U.S. Pat. No. 5,827,740; Jaiswal, N., et al., J. Cell Biochem. (1997) 64(2): 295 312; Cassiede P., et al., J. Bone Miner. Res. (1996) 11(9):1264 1273; Johnstone, B., et al., (1998) 238(1):265 272; Yoo, et al., J. Bone Joint Sure. Am. (1998) 80(12):1745 1757; Gronthos, S., Blood (1994) 84(12): 41644173; and Makino, S., et al., J. Clin. Invest. (1999) 103(5):697-705).

Although cells in accordance with the invention are typically culturally expanded prior to use, it is also possible to use such cells without culture expansion. For example, mesenchymal stem cells may be derived from bone marrow and used after separation of the MSCs from the blood cells, without expansion. Thus, for example, bone marrow may be enriched in human mesenchymal stem cells by removal of blood cells, and introduced into a subject as described herein.

Cells that may be useful in methods of the present invention may have properties of stem cells or progenitor cells, such as MSCs, HSCs, progenitor cells, or the like. These cells may subsist within the area of administration to the subject, such as the vitreous of the eye or the CSF, and remain for extended periods of time. For example, cells introduced into the subject may survive for about 1 week, about 2 weeks, about 1 month, about 2 months, about 3 months, about 4 months, about 5 months, about 6 months, about 7 months, about 8 months, about 9 months, about 10 months, about 11 months, about 12 months, about 18 months, about 2 years, or about 5 years, or longer. In an embodiment, such cells may survive in the area of administration indefinitely. In embodiments where the cells are injected into the vitreous of the eye, such cells may remain within the vitreous, or they may integrate into the retina.

In an embodiment, the invention provides a composition comprising a population of cells or a plurality of cells producing, expressing, or overexpressing a therapeutic compound genetically engineered to produce and release molecules that can have therapeutic benefit for the organs or tissues into which they are implanted. Such cells may be produced by transduction or transformation of a population of cells with a construct that directs stable expression or synthesis of the therapeutic agent to be delivered. The expression can be constitutive or inducible by drug administration. In one embodiment, the DNA construct of the invention may be delivered by a vector such as an adeno-associated viral vector and may contain a functional gene encoding a protein of interest. Methods for transduction or transformation of cells are known in the art. In one embodiment, the protein may be a therapeutic polypeptide, for example TPP1. Such cells may be useful in accordance with the invention for treatment of a retinal degeneration disease such as CLN2 or the like. As a result of said treatments or cell manipulation to overexpress a therapeutic polypeptide, the cells of the cell population produce and release a compound such as a protein or peptide that can be used in the treatment of a neurodegenerative disease, a retinal degenerative disease, or a disease causing degenerative changes in the CNS in accordance with the invention. To that end, a cell population as described herein may be implanted or introduced into the eye of a subject in need of treatment of a retinal degeneration disease or into the CNS for treatment of a neurodegenerative disease.

As used herein, a cell that expresses or overexpresses a therapeutic protein such as TPP1 may be a cell, such as a cell selected from the group consisting of an MSC, an HSC, a progenitor cell, or the like, that has been genetically manipulated to produce and release a compound that has a therapeutic benefit. In an embodiment, the DNA construct directing synthesis of such a polynucleotide encoding a therapeutic polypeptide may be comprised in an expression cassette, and said polynucleotide may be operatively bound to (i.e., under the control of) an expression control sequence. Expression control sequences are sequences that control and regulate transcription and, where appropriate, translation of a protein, and include promoter sequences, sequences encoding transcriptional regulators, ribosome binding sequences (RBS), and/or transcription terminator sequences. In a particular embodiment, said expression control sequence is functional in eukaryotic cells, such as animal or mammalian cells, for example, the human cytomegalovirus (hCMV) promoter, the combination of the cytomegalovirus (CMV) early enhancer element and chicken beta-actin promoter (CAG), the eukaryotic translation initiation factor (elF) promoter, or the like. One of skill will recognize that any suitable combination of expression control sequences may be used in accordance with the invention.

Said expression cassette may further comprise a marker or gene encoding a motif or producing a phenotype allowing the selection of the host cell transformed or transduced with said expression cassette. Illustrative examples of said markers that could be present in the expression cassette of the invention include antibiotic-resistant genes, toxic compound-resistant genes, fluorescent marker-expressing genes, and generally any genes that allow selecting the genetically transformed or transduced cells. The gene construct can be inserted in a suitable vector. The choice of the vector will depend on the host cell where it will subsequently be introduced. By way of illustration, the vector in which the polynucleotide comprising the nucleotide sequence encoding the functional TPP1 protein is introduced can be a plasmid or an adeno-associated viral vector which, when introduced in a host cell, either becomes integrated or not in the genome of said cell. Said vector can be obtained by conventional methods known by persons skilled in the art [Sambrook and Russell, “Molecular Cloning, A Laboratory Manual,” 3rd ed., Cold Spring Harbor Laboratory Press, N.Y., 2001 Vol 1-3]. In a particular embodiment, said recombinant vector is a vector that is useful for transforming or transducing animal cells, preferably mammalian cells. Said vector can be used to transform, transfect, transduce, or infect cells such as cells selected from the group consisting of MSCs, HSCs, progenitor cells, and the like. Transformed, transfected, transduced, or infected cells can be obtained by conventional methods known by persons skilled in the art [Sambrok and Russell, (2001), cited supra].

The cells for use in the present invention, for instance for use in treatment of a neurodegeneration or retinal degeneration disease according to the invention, may be any cell type capable of undergoing the necessary genetic modification, and may be isolated cells. Such cells may be used to initiate, or seed, cell cultures. The specific cells may be isolated in view of their markers as previously described. Isolated cells may be transferred to sterile tissue culture vessels, either uncoated or coated with extracellular matrix or ligands such as laminin, collagen (native, denatured, or crosslinked), gelatin, fibronectin, and other extracellular matrix proteins. The cells for use in the present invention may be cultured in any suitable culture medium (depending on the nature of the cells) capable of sustaining growth of said cells such as, for example, DMEM (high or low glucose), advanced DMEM, DMEM/MCDB 201, Eagle B basal medium, HamS F10 medium (F10), HamS F-12 medium (F12), Iscove's modified Dulbecco's-17 medium, DMEM/F12, RPMI 1640, or the like. If necessary, the culture medium may be supplemented with one or more components including, for example, fetal bovine serum (FBS); equine serum (ES); human serum (HS); beta-mercaptoethanol (BME or 2-ME); one or more growth factors, for example, platelet-derived growth factor (PDGF), epidermal growth factor (EGF), fibroblast growth factor (FGF), vascular endothelial growth factor (VEGF), insulin-like growth factor-1 (IGF-1), leukocyte inhibitory factor (LIF), stem cell factor (SCF) and erythropoietin; cytokines as interleukin-3 (IL-3), interleukin-6 (IL-6), FMS-like tyrosine kinase 3 (Flt3); amino acids, including L-valine; and one or more antibiotic and/or antimycotic agents to control microbial contamination, such as, for example, penicillin G, streptomycin sulfate, amphotericin B, gentamicin, or nystatin, either alone or in combination. The cells may be seeded in culture vessels at a density to allow cell growth.

Methods for the selection of the most appropriate culture medium, medium preparation, and cell culture techniques are well known in the art and are described in a variety of sources, including Doyle et al., (eds.), 1995, Cell & Tissue Culture: Laboratory Procedures, John Wiley & Sons, Chichester; and Ho and Wang (eds.), 1991, Animal Cell Bioreactors, Butterworth-Heinemann, Boston.

### Kits

In still further embodiments, the present invention provides kits for use with the methods described herein for treatment of a neurodegenerative disease in a subject, for instance, comprising at least one cell expressing a therapeutic compound for treating a neurodegenerative disease in the subject, such as a canine, a feline, a mouse, or a human. The kit may include one or more sterile containers into which the tissue containing the desired cell population is placed. The container, for example, may be a vial, a tube, a flask, or a syringe.

In further embodiments, the kit includes one or more tubes or wells of a culture or microtiter plate or other cell sterile cell culture plates or flasks into which autologous cells may be placed. The kit may allow for the assay of a single sample, or more than one sample. In some embodiments, the kit includes a plurality of plates or tubes, which allow for numerous samples concurrently or consecutively. In particular embodiments, the autologous cells may be bone marrow-derived mesenchymal stem cells.

The treatment reagents of the kit may take any one of a variety of forms, including reagents with which to grow cells in culture. Such reagents may include, but are not limited to, any cell culture reagents known in the art and useful with the methods of the invention, for example, fetal bovine serum, antibiotics, or the like. Detection assays that are associated with and/or linked to a given therapeutic agent or protein may be included in a kit according to the invention. Detectable labels that are associated with and/or attached to a secondary binding ligand are also contemplated. Exemplary secondary ligands are those secondary antibodies that have binding affinity for the first antibody. A number of exemplary assays are known in the art and/or all such assays may be employed in connection with the present invention. Assays for some therapeutic compounds envisioned may be performed at certified analytical laboratories. When this is the case, the kits may include instructions and materials for submitting samples to the appropriate certified labs for analysis.

The kits may optionally include a suitably aliquoted composition of a therapeutic agent of the invention to serve as a positive control. The components of the kits may be packaged either in aqueous media and/or in lyophilized form, and may be suitable for storage at any temperature. In another embodiment, kits of the present invention may comprise instructions or written directions for use of the kit.

### DEFINITIONS

As used herein, “autologous” refers to a cell or cell type that is derived from the same individual who is to be treated. For example, in accordance with the invention, an autologous cell is a cell or a sample of cells that are isolated from an individual subject (e.g., a canine or human), transduced with a construct comprising a polynucleotide sequence encoding a protein of interest, expanded in culture to create a population of cells all of which comprise the construct, and injected back into the same subject for treatment.

As used herein, a “neurodegenerative disease” refers to a disease that results in progressive loss of structure or function of neurons, including death of neurons. Such a disease may exhibit a number of symptoms, including, but not limited to, visual impairment, complete blindness, seizures, loss of motor and/or cognitive function, and the like. A neurodegenerative disease may in one embodiment include a retinal degenerative disease. A neurodegenerative disease in accordance with the invention may encompass a CNS degenerative disease, a retinal degeneration disease, a lysosomal storage disease, a neuronal ceroid lipofuscinosis disease, or other diseases producing similar symptoms or having a common etiology. In some cases, orthologous genes in different species may exhibit similar mutations, thus resulting in similar diseases between species. For this reason, some species may serve as useful models for disease in other species.

As used herein, a “retinal disorder” refers to a defect in the tissue of the retina. Retinal disorders may result from mutations in specific genes, infection, injury, or a degenerative condition related to aging, metabolic disease, or environmental factors. A retinal disorder may include any condition that leads to the impairment of the retina's normal function. A “retinal degeneration disease” in accordance with the invention is a disease associated with deterioration of the retina caused by the progressive and eventual death of the cells of the retinal tissue. The term “retinal degeneration disease” also includes indirect causes of retinal degeneration, i.e., retinal degenerative conditions derived from other primary pathologies, such as cataracts, diabetes, glaucoma, or the like. In a particular embodiment, said retinal degeneration disease is selected from the group comprising retinitis pigmentosa, age-related macular degeneration, diabetic retinopathy, Stargardt disease, cone-rod dystrophy, congenital stationary night blindness, Leber congenital amaurosis, Best's vitelliform macular dystrophy, anterior ischemic optic neuropathy, choroideremia, age-related macular degeneration, foveomacular dystrophy, Bietti crystalline corneoretinal dystrophy, Usher S syndrome, etc., as well as retinal degenerative conditions derived from other primary pathologies, such as cataracts, diabetes, glaucoma, etc. In another particular embodiment, said retinal degeneration disease is age-related macular degeneration that is presented in two forms: “dry” that results from atrophy to the retinal pigment epithelial layer below the retina, which causes vision loss through loss of photoreceptors (rods and cones) in the central part of the eye; and “wet” that causes vision loss due to abnormal blood vessel growth (choroidal neovascularization) in the choriocapillaris, through BruchS membrane, ultimately leading to blood and protein leakage below the macula, eventually causing irreversible damage to the photoreceptors and rapid vision loss. In a more particular embodiment, said retinal degeneration disease is RP, a heterogeneous family of inherited retinal disorders characterized by progressive degeneration of the photoreceptors with subsequent degeneration of RPE, which is characterized by pigment deposits predominantly in the peripheral retina and by a relative sparing of the central retina. In most of the cases of RP, there is primary degeneration of photoreceptor rods, with secondary degeneration of cones.

The phrase “treating a retinal degenerative disease” refers to ameliorating the effects of, or delaying, halting or reversing the progress of, or delaying or preventing the onset of, a retinal degenerative disease as defined herein.

As used herein, a “therapeutic compound” refers to a molecule, such as a protein, peptide, polypeptide, a carbohydrate, an antibody or antibody fragment, a small molecule, or the like, that is not functionally present in cells of the subject or the like, and/or that will have a therapeutic benefit when delivered to cells of the subject.

As used herein, “subject” or “patient” refers to animals, including mammals, who are treated with the pharmaceutical compositions or in accordance with the methods described herein.

As used herein, “pharmaceutically acceptable carrier,” “carrier,” “medium,” or “biologically compatible carrier” refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio.

As used herein, “cell culture,” “in culture,” or “cultured” refers generally to cells taken from a living organism and grown under controlled conditions. A “primary cell culture” is a culture of cells, tissues, or organs taken directly from an organism before the first subculture. Cells are expanded in culture when they are placed in a growth medium under conditions that facilitate growth and/or division, resulting in a larger population of the cells. When cells are expanded in culture, the rate of cell proliferation is sometimes measured by the amount of time needed for the cells to double in number, referred to as “doubling time.”

As used herein, a “cell line” is a population of cells cultured in vitro formed by one or more subcultivations of a primary cell culture. Each round of sub-culturing is referred to as a passage. When cells are subcultured, they are referred to as having been passaged. A specific population of cells, or a cell line, is sometimes referred to or characterized by the number of times it has been passaged For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be under stood by those of skill in the art that there may be many population doublings during the period of passaging; there fore the number of population doublings of a culture is greater than the passage number. The expansion of cells (i.e., the number of population doublings) during the period between passaging depends on many factors, including but not limited to the seeding density, substrate, medium, growth conditions and time between passaging.

“Mesenchymal cells” are mesodermal germ lineage cells and may or may not be differentiated. The mesenchymal cells of the invention include cells at all stages of differentiation beginning with multipotent mesodermal germ cells, down to fully differentiated terminal cells. Examples of mesenchymal cells which are terminal cells include, but are not limited to, endothelial cells, fibroblasts, osteoblasts, chondrocytes, myocytes, and adipocytes. The mesenchymal cells of the invention may be multipotent stem cells capable of forming multiple cells belonging to the mesodermal lineage (i.e. multipotent mesenchymal stromal cells or “MMSCs”). Mesenchymal cells may also be regenerative precursor cells capable of dividing and differentiating into a specific terminal cell.

As used herein, “introducing,” “delivering,” and “administering” refer to the therapeutic introduction of autologous mesenchymal cells or other cells to a subject. Administration may take place by any route that allows the cells to treat a retinal degenerative disorder in accordance with the invention. The cells may be directly administered to the eye of the patient through a variety of modes including, but not limited so to, retrobulbar injection, intravitreous injection, and subchoroidal injection.

“Retinal tissue” refers to the neural cells and associated vasculature that line the back of the eye. Structures within retinal tissue include the macula and fovea. Retinal tissue further includes the tissue that is juxtaposed to these neural cells (e.g. pigment epithelia) and associated vasculature.

As used herein, “stem cells” refers to undifferentiated cells defined by the ability of a single cell both to self-renew, and to differentiate to produce progeny cells, including self-renewing progenitors, non-renewing progenitors, and terminally differentiated cells. Stem cells are also characterized by their ability to differentiate in vitro into functional cells of various cell lineages. Stem cells may have varying degrees of potency. Pluripotent stem cells are capable of giving rise to cells belonging to each of the three embryonic germ layers (i.e. the endoderm, mesoderm and ectoderm). Multipotent stem cells are more lineage-restricted than pluripotent stem cells as they are only capable of forming cells from a single lineage (e.g. ectodermal cells). Stem cells may also be progenitor cells (i.e. precursor cells) which are lineage-committed cells capable of both dividing and differentiating into a specific terminal cell type. Fetal neural stem cells are derived from the neural tissue of a mammalian fetus after at least 7 to 12 weeks of gestation. In the case of humans, fetal neural stem cells are typically but not isolated exclusively between 7 and 12 weeks of gestation. A stem cell in accordance with the invention may be any type of stem cell, including, but not limited to, a hematopoietic stem cell (HSC), a progenitor cell, a mesenchymal stem cell (MSC), an embryonic stem cell (ESC), an embryoid body, or the like. One of skill will understand that various types of cells may be used and will understand how to use such cells in accordance with the invention. In some embodiments, one type of stem cell may be converted to another type of cell or stem cell. One of skill will be able to identify such appropriate techniques to achieve this purpose, for example cell culture techniques may used to convert one type of cell into another. In another particular embodiment the progenitor cell may be a lineage-restricted cell.

As used herein, “multipotent” or “multipotency” refers to the ability of a stem cell to differentiate into various cells of one embryonic germ layer lineage (i.e the ectoderm, endoderm or meso-derm).

As used herein, “pluripotent” or “pluripotency,” refers to the ability of a stem cell to differentiate into the various cells from each of the so three embryonic germ layer lineages (i.e the ectoderm, endo-derm and mesoderm).

Stem cells may also be categorized on the basis of their source. An adult stem cell is generally a multipotent undifferentiated cell found in tissue comprising multiple differentiated cell types. An adult stem cell has the ability to renew itself. Under normal circumstances, such a cell can also differentiate to yield the specialized cell types of the tissue from which it originated, and possibly those obtained from umbilical cord blood or non-blood-derived (e.g., as obtained from the non-blood tissues of the umbilical cord and placenta).

As used herein, a “hematopoietic stem cell” or “HSC” refers to a multipotent stem cell that gives rise to all the blood cell types from the myeloid (monocytes and macrophages, neutrophils, basophils, eosinophils, erythrocytes, megakaryocytes/platelets, dendritic cells), and lymphoid lineages (T-cells, B-cells, NK-cells). HSCs are a heterogeneous population of cells.

As used herein, the terms “precursor cell,” “tissue precursor cell” and “progenitor cell” are used interchangeably herein and refer to a lineage-committed cell that divides and differentiates to form new, specialized tissue(s). Endothelial precursor cells are one example of a precursor cell.

As used herein, “regenerative” refers to the ability of a substance to restore, supplement, or otherwise rehabilitate the natural function of a tissue. This ability may be conferred by, for example, treating a dysfunctional tissue with regenerative cells. Regenerative cells treat dysfunctional tissue by replacing it with new cells capable of performing the tissue's natural function, or by helping to restore the natural activity of dysfunctional tissue.

The terms “restore,” “restoration” and “correct” are used interchangeably herein and refer to the regrowth, augmentation, supplementation, and/or replacement of a defective tissue with a new and preferentially functional tissue. The terms include the complete and partial restoration of a defective tissue. Defective tissue is completely replaced if it is no longer present following the administration of the inventive composition. Partial restoration exists where defective tissue remains after the inventive composition is administered.

The phrase “effective amount” refers to a concentration or amount of a reagent, pharmaceutical composition, protein, cell population or other agent, that is effective for producing intended result, including treatment of retinal degenerative or neurodegenerative conditions, cell growth and/or differentiation in vitro or in vivo as described herein. With respect to the administration of one or more populations of regenerative cells as disclosed herein, an effective amount may range from as few as several hundred or fewer to as many as several million or more. It will be appreciated that the number of cells to be administered will vary depending on the specifics of the disorder to be treated, including but not limited to size or total volume/surface area to be treated, as well as proximity of the site of administration to the location of the region to be treated, among other factors familiar to one of skill.

As used herein, “functionally present” refers to a form or variant of a therapeutic compound as described herein, in which the compound is not functional in the subject or cell. For example, a cell or subject may comprise a specific gene, gene sequence, protein, polypeptide, or the like, but that gene, gene sequence, protein, polypeptide, or the like is non-functional, absent, or otherwise inactive. The methods and compositions of the present invention may provide a supplemental or replacement form or variant of such a compound in order to restore or provide the intended function of the compound.

A “clone,” or “clonal cell,” is a line of cells that is genetically identical to the originating cell. This cloned line is produced by cell division (mitosis) of the originating cell. The term “clonal population” in reference to the cells of the invention shall mean a population of cells that is derived from a clone. A cell line may be derived from a clone and is an example of a clonal population.

Endothelial precursor cells are one example of a mesenchymal cell. The mesenchymal cells of the invention may be derived from sources including umbilical cord blood, placenta, Wharton's jelly, bone marrow, chorionic villus, adipose tissue, menstrual discharge, amniotic fluid and peripheral blood, and combinations thereof. Mesenchymal cells may also be derived from the in vitro differentiation of pluripotent embryonic stem cells.

In a broad sense, a progenitor cell is a cell that has the capacity to create progeny that are more differentiated than itself, and yet retains the capacity to replenish the pool of progenitors. By that definition, stem cells themselves are also progenitor cells, as are the more immediate precursors to terminally differentiated cells. When referring to the cells of the present invention, as described in greater detail below, this broad definition of progenitor cell may be used. In a narrower sense, a progenitor cell is often defined as a cell that is intermediate in the differentiation pathway, i.e., it arises from a stem cell and is intermediate in the production of a mature cell type or subset of cell types. This type of progenitor cell is generally not able to self-renew. Accordingly, if this type of cell is referred to herein, it will be referred to as a non-renewing progenitor cell or as an intermediate progenitor or precursor cell

### EXAMPLES

The following examples are included to demonstrate preferred embodiments of the invention. It should be appreciated by those of skill in the art that the techniques disclosed in the examples, which follow represent techniques discovered by the inventor to function well in the practice of the invention, and thus can be considered to constitute preferred modes for its practice. However, those of skill in the art should, in light of the present disclosure, appreciate that many changes can be made in the specific embodiments, which are disclosed and still obtain a like or similar result without departing from the spirit and scope of the invention.

### Example 1

### Development of a Research Colony of Dachshunds with CLN2

Testing was performed on a Dachshund that died as a result of neuronal ceroid lipofuscinosis (NCL) (Awano et al., Mol. Genet. Metabol. 89, 254-260, 2006). Based on the ultrastructural appearance of the lysosomal storage material that accumulated in neurons of the dog, it appeared likely that the form of NCL from which it suffered was analogous to human late infantile neuronal ceroid lipofuscinosis (CLN2 or LINCL). The canine ortholog of the TPP1 gene was sequenced using DNA from this dog (GenBank Accession No. NM_001013847.1). Sequence analyses revealed a single nucleotide deletion on exon 4 of TPP1 that predicted a frame shift and premature stop codon (Awano et al., Mol. Genet. Metabol. 89, 254-260, 2006). Both parents of the affected dog were located and obtained, and both were found to be heterozygous for the deletion mutation. The heterozygous dogs were bred to each other, and their first litter consisted of two puppies that were heterozygous for the mutation and two that were homozygous for the mutant allele. Both homozygous dogs developed NCL and died at approximately 10.5 months of age. The heterozygous dogs remained healthy with no neurological abnormalities.

Using the original breeding pair of Dachshunds, their descendents, and unrelated homozygous normal dogs, subsequent breedings have been conducted to develop a research colony that has been used to characterize the disease phenotype and to assess therapeutic interventions. Over 30 dogs, including carrier males and females and a few outside unrelated homozygous normal dogs have rotated through the breeding colony. The current breeding colony consists 9 carrier females and 6 carrier males as well as frozen semen from 5 homozygous affected males. Most breedings are performed between carriers. Affected dogs do not live beyond 11 months of age. Because sexual maturity in this breed usually does not occur until 9 to 12 months of age, affected females cannot be used for breeding. In some cases, affected males reach sexual maturity before becoming severely debilitated, particularly if they have been treated with enzyme replacement or gene therapy. When this occurs semen is collected from the affected male, evaluated for quality, and if of sufficient quality is preserved using standard techniques. Then when a female is in heat, semen from the affected male is bred with the carrier female. To keep the colony from becoming too inbred, unrelated normal Dachshunds are periodically incorporated into the breeding program. All puppies were implanted with microchips and genotyped for the TPP1 mutation using an allelic discrimination assay.

All of the dogs generated from this breeding colony that were homozygous for the TPP1 deletion mutation developed NCL whereas none of the heterozygous dogs or dogs homozygous for the normal allele developed the disease.

### Example 2

### Demonstration of Efficacy of TPP1 Enzyme Replacement Therapy and TPP1 Gene Therapy for the Brain

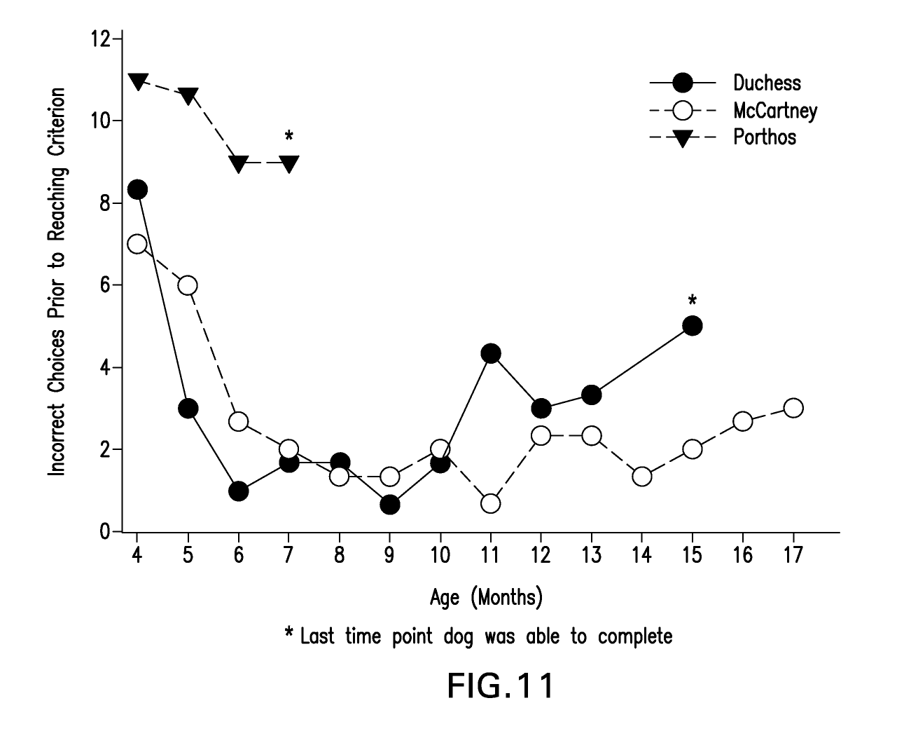

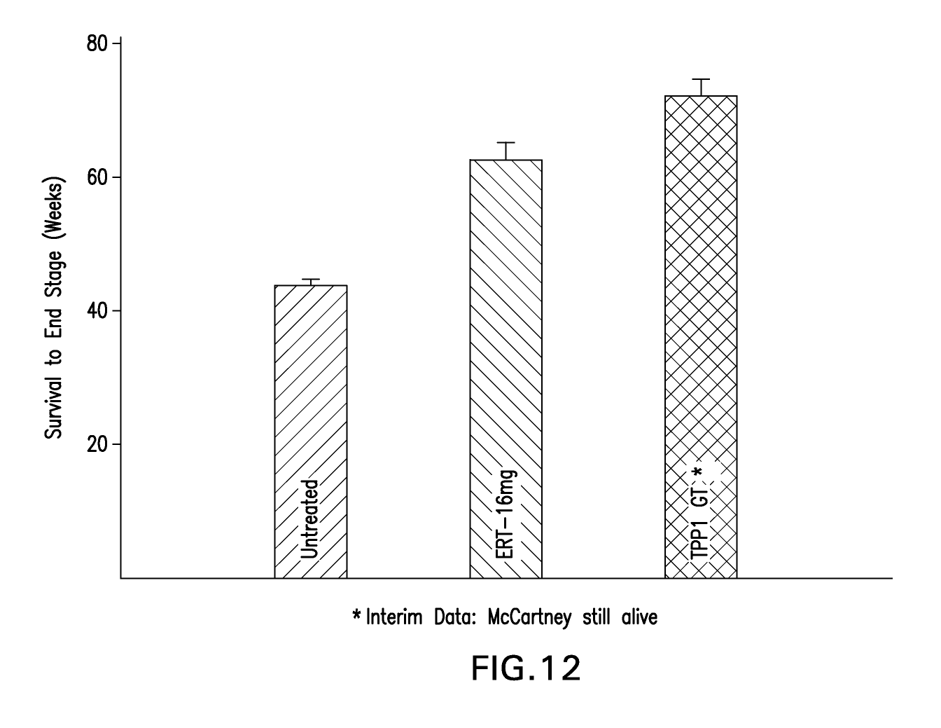

In the canine model, a frameshift mutation in the canine ortholog of TPP1 results in disease symptoms very similar to human CLN2. Homozygous dogs have very little or no TPP1 activity and thus suffer from progressive neurodegeneration that is very similar to human CLN2. Using the Dachshund CLN2 model, studies were conducted to determine whether administration of pro-TPP1 to the CNS prevents or delays the disease-related neurodegeneration and accompanying neurological signs. Periodic infusion of recombinant human TPP1 (rhTPP1) into the cerebrospinal fluid (CSF) of affected dogs significantly delayed brain degeneration and progression of neurological signs, while also increasing longevity (Katz et al., J Neurosci Res 92:1591-1598, 2014). One-third of the CLN2-affected dogs that received TPP1 replacement therapy showed a delay in the onset of pupillary light reflex (PLR) deficits. PLR constriction amplitudes remained normal through 10 months of age while deficits first appeared at 6 months of age in untreated dogs. Even at 12 months of age, deficits were minor compared with constriction amplitudes of untreated dogs (Whiting et al., Exp Eye Res 125:164-172, 2014). CSF infusions of TPP1 were not effective in preventing loss of retinal function, suggesting that TPP1 infused into the CSF does not reach most of the retina. Directed delivery of TPP1 to the retina may therefore halt retinal degeneration and preserve vision in affected dogs. Similar therapeutic benefits were observed from a single administration of a TPP1 gene therapy vector to the CSF (see FIGS. 11 and 12).

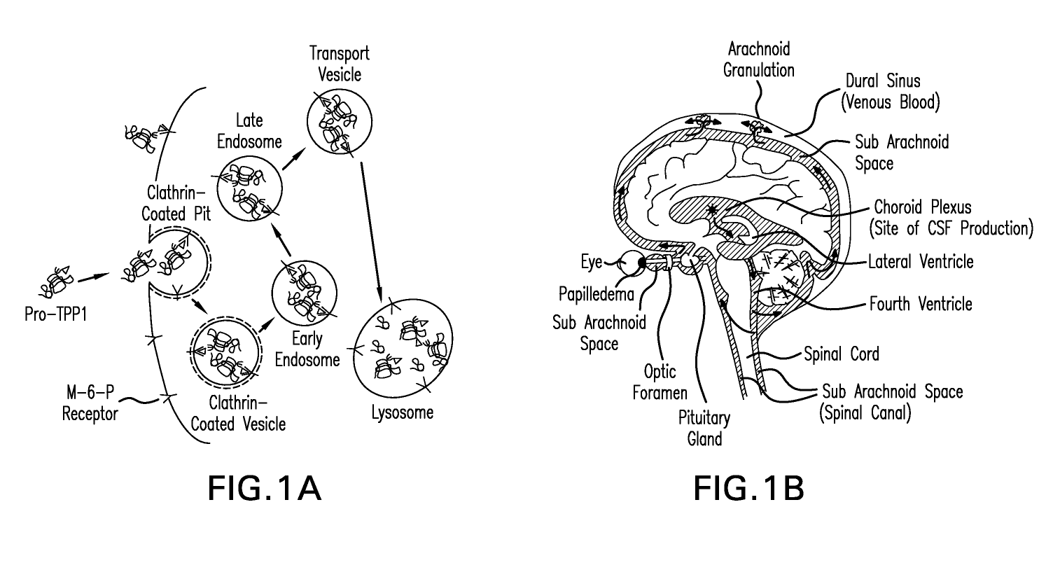

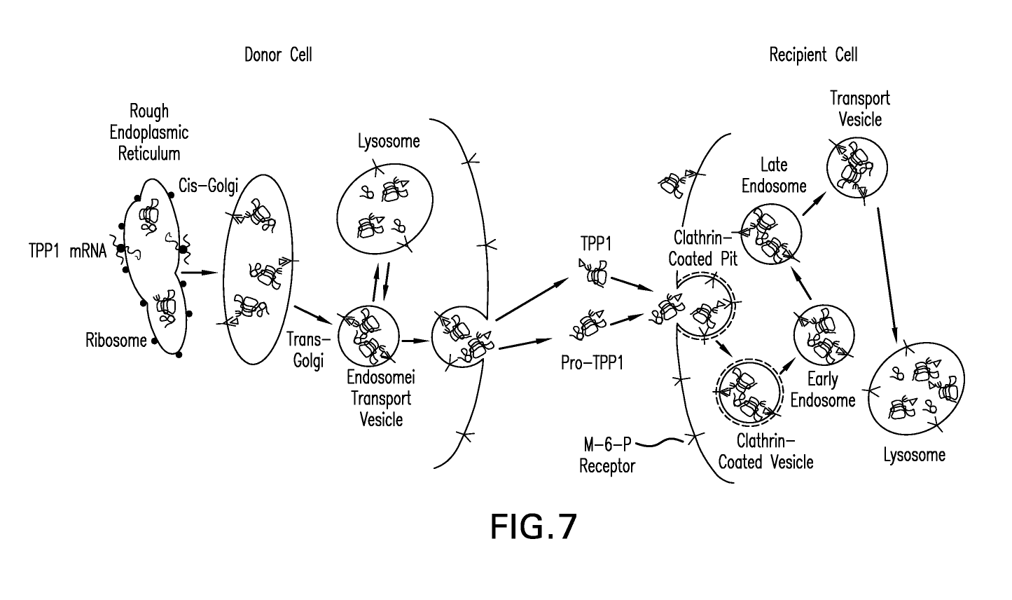

Pro-TPP1 or mature TPP1 external to the cell binds to mannose-6-phosphate receptors on the cell surface, and is transported to the lysosomes via clathrin-coated vesicles and the cellular endosomal system (see, for example, FIG. 7). Upon fusion of endosome containing the TPP1 or pro-TPP1 with a lysosome, the protein is released from the membrane-bound receptor into the lysosome lumen and, if in the pro-form, is activated by proteolytic cleavage to a mature form (Whiting et al., Exp Eye Res 125:164-172, 2014). In both enzyme replacement studies and direct gene therapy studies, widespread distribution of the pro-enzyme into the CNS was achieved by taking advantage of the fact that the CSF circulates around and bathes the entire CNS (FIG. 1B). In an initial short-term study, affected dogs were given 4 bolus injections of recombinant human pro-TPP1 (rhTPP1) into the cisterna magna at 2-week intervals. The dogs were euthanized 48 hours after the last injection, and a variety of brain regions were analyzed for the amounts of active TPP1 enzyme and of the autofluorescent storage material that accumulates in this disease. TPP1 activity was detected in every region of the brain examined and, in many regions, the enzyme activity was a significant fraction of normal levels (Vuillemenot et al., Mol. Genet. Metabol. 104, 325-337, 2011). Likewise, the amounts of storage material in many brain regions were reduced by the treatment.

The bolus injections, however, resulted in immune-mediated anaphylaxis, most likely due to the fact that high peak CSF levels of TPP1 resulting from bolus injections resulted in substantial release of the TPP1 into the systemic circulation (Vuillemenot et al., Molec Genet Metabol, first published online 16 Sep. 2014). As a consequence, in subsequent enzyme replacement studies, the rhTPP1 was administered by slow infusion into the CSF via an implanted catheter. The treatment protocol was optimized such that any detectable immune reaction to the recombinant protein was minimal. The protein was thus administered into the CSF, in increasing doses, at 2-week intervals until a target dosage was achieved. Using this approach, widespread distribution of active rhTPP1 to most brain regions was achieved, which significantly delayed the onset and progression of neurological signs and neurodegeneration (Katz et al., J Neurosci Res 92:1591-1598, 2014). Similar results have been obtained by injecting an AAV2-CAG-TPP1 vector into the CSF via a lateral ventricle of the brain at an early stage of the disease progression (see FIGS. 11 and 12). This resulted in continuous release of functional TPP1 into the CSF throughout the lives of the treated dogs.

These studies demonstrate that supplying pro-TPP1 to CSF in contact with most brain structures results in uptake of TPP1 protein into the brain parenchyma and its activation in parenchymal cell lysosomes. Providing the TPP1 via the CSF over an extended period results in dramatic delays in a number of measures of disease progression related to brain structure and function. Unfortunately, long-term delivery of TPP1 via the CSF, either by infusion of rhTPP1 or via injection of a TPP1 expression gene construct did not prevent retinal degeneration or impairment of retinal function, perhaps because the CSF circulation does not reach the retina.

### Example 3

### Characterization of Retinal Degeneration in a Canine Model for CLN2

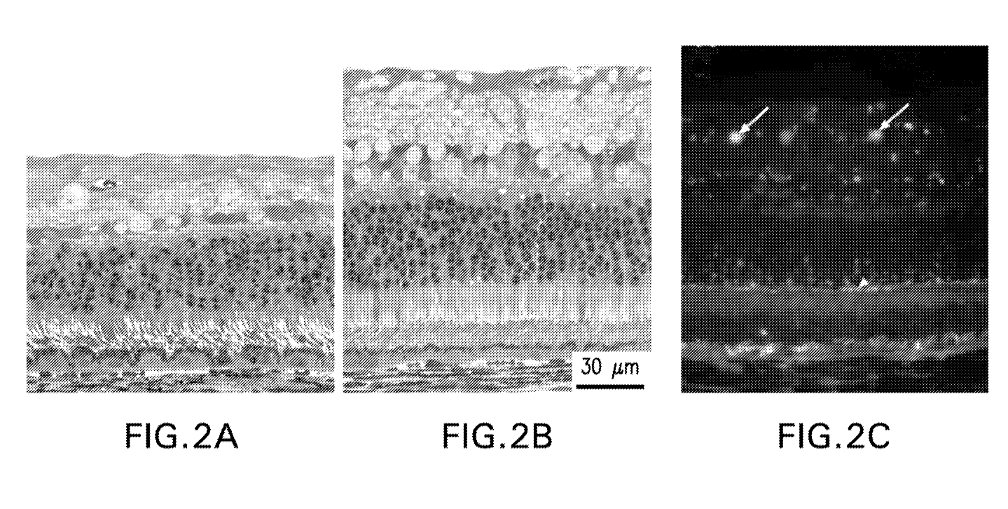

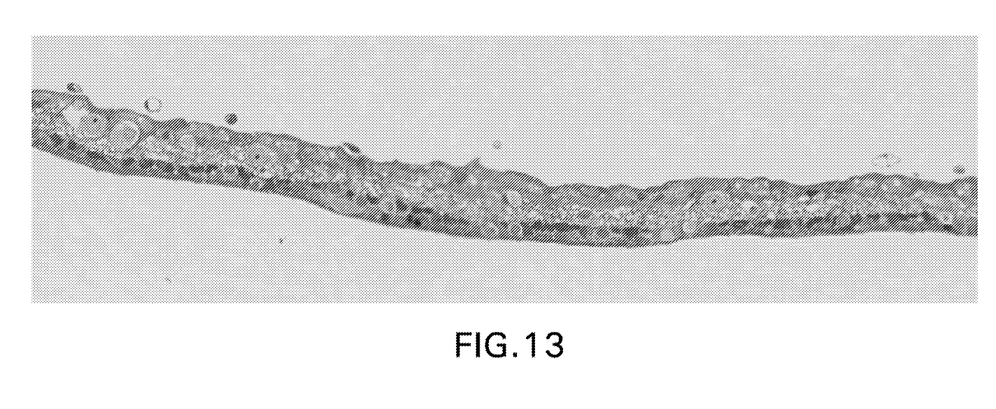

In addition to causing progressive brain atrophy and neurological decline, the lack of TPP1 enzyme in the CLN2 dogs is accompanied by progressive retinal degeneration and the accumulation of autofluorescent lysosomal storage bodies in most layers of the retina (Katz et al., Invest. Ophthalmol. Vis. Sci. 49, 2686-2695, 2008) (FIG. 2). In untreated affected dogs, retinal degeneration is characterized primarily by thinning of the inner retinal layers, with the photoreceptor layer being affected to a much lesser extent (FIG. 2). However, in dogs that were treated with CSF delivery of TPP1 and therefore survived for longer periods of time, retinal degeneration progressed to the point that almost the entire retina degenerated (FIG. 13).

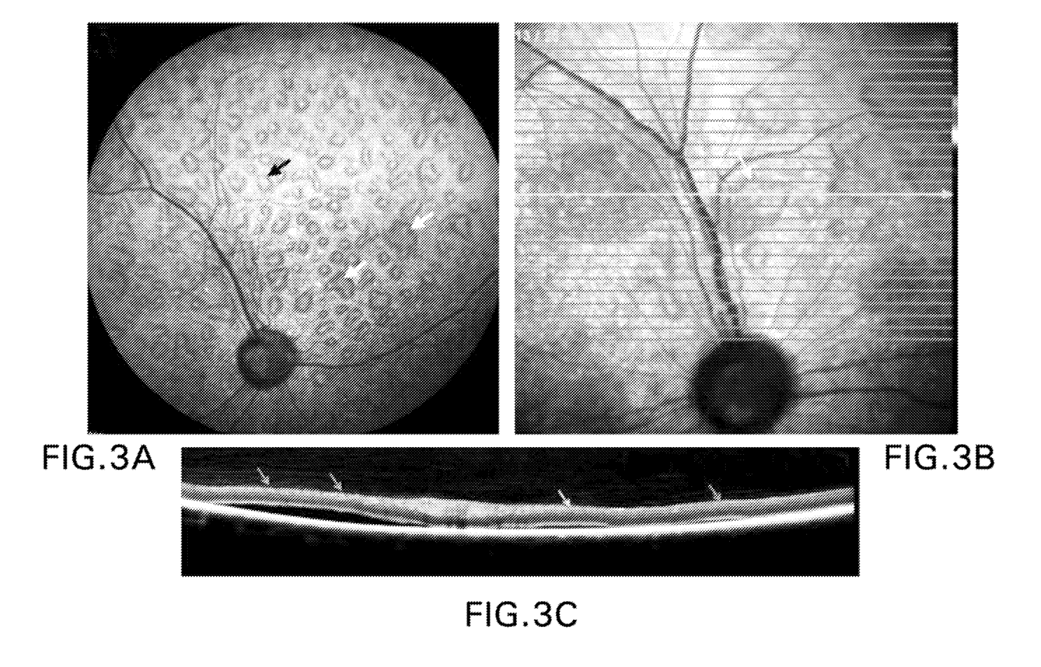

Using scanning laser ophthalmoscopy (SLO) to obtain fundus images of the retina, the affected dogs were found to develop unusual retinal lesions that are somewhat similar to those reported in Best vitelliform macular dystrophy (Chacon-Camacho et al., Ophthalmic Genetics 32, 24-30, 2011) (FIG. 3A). These lesions can begin to appear as early as 5 months of age. Initially they are few in number and widely scattered in the retina, and over time the numbers and sizes of the lesions increase dramatically. By disease end-stage at 10 to 11 months of age, the lesions can occupy a significant fraction of the retina (FIG. 3A). Optical computed tomography (OCT) imaging of these lesions indicated that they were localized retinal detachments where the neural retina had separated from the retinal pigment epithelium (RPE) (FIGS. 3B and 3C). These lesions were present only in the dogs that were homozygous for the TPP1 null mutation and were not observed in either heterozygotes or in dogs homozygous for the normal TPP1 allele. No mutations were found in the coding region or exon/intron junctions of the BEST1 gene in dogs exhibiting these lesions. Therefore, these lesions develop as a consequence of the TPP1 mutation, and thus, inhibition of their development can be used as a marker for therapeutic efficacy.

Administration of rhTPP1 to the CSF starting at 2 months of age did not prevent these disease-related changes in the retina, nor did administration of the AAV2-CAG-TPP1 gene expression vector to the CSF. The pathological changes in the retinas were accompanied by progressive impairment of retinal function as assessed by electroretinogram (ERG) measurements (Katz et al., Invest. Ophthalmol. Vis. Sci. 49, 2686-2695, 2008; Whiting et al., Exp Eye Res 125:164-172, 2014). Prior to 4 months of age, the ERG amplitudes and waveforms were essentially normal in dogs that were homozygous for the TPP1 null mutation. However, by 6 months of age, the affected dogs showed substantial decreases in b-wave amplitudes, which continued to decline as the disease progressed (Whiting et al., Exp Eye Res 125:164-172, 2014). A-wave amplitudes were also depressed in the affected dogs, but to a much lesser degree that the b-wave amplitudes (Whiting et al., Exp Eye Res 125:164-172, 2014). The A-wave is generated by the photoreceptor cells and the b-wave by cells of the inner retina, so the preferential decline in b-wave amplitudes was consistent with the histological data showing that degenerative changes were much more pronounced in the inner retina than in the photoreceptor cell layer.

The PLR is the change in pupil size that occurs in response to changes in ambient light levels, and is mediated by responses from both the photoreceptor cells and photosensitive ganglion cells (Sand et al., Progress in Retinal & Eye Research 31, 287-302, 2012). Instrumentation and procedures were developed for quantitative assessment of the PLR in dogs. As expected from the histological and ERG findings, PLR amplitudes were greatly reduced in dogs that were homozygous for the TPP1 null mutation. This deficit can first be measured around 6 months of age and continues to progress through end-stage disease (Whiting et al., 2013). However, surprisingly, administration of rhTPP1 to the CSF starting at 2 months of age almost completely prevented the disease-related decline in the PLR (Whiting et al., Exp Eye Res 125:164-172, 2014). The mechanism for this protective effect has not been determined, but it is likely the result of delivery of rhTPP1 from the CSF to the brain regions involved in the PLR (the pretectal and Edinger-Westphal nuclei) as well as to the ganglion cells via the CSF that bathes the optic nerve (FIG. 1B).

Delivery of TPP1 to the CSF is therefore therapeutically effective in delaying disease progression in the CNS but not the retina. In order to preserve retinal function, direct delivery of TPP1 to the eye will be required. This may be achieved by the means proposed in this patent application, a means that can also be applied as an alternative for continuous delivery of TPP1 (or other therapeutic substance) to the CNS

### Example 4

### Demonstration of Long-Term Survival of MSCs Implanted into the Mouse Vitreous

Implantation of neutralized embryonic stem cells from normal mice into the vitreous of the eyes of mice undergoing slow retinal degeneration was previously found to inhibit degeneration of the host retina (Meyer et al., Stem Cells 24, 274-283, 2006). The donor cells remained mostly at the vitreal surface of the retina, but there was some integration of donor cells into the host retina. These studies indicated that implanted donor cells can have a neuroprotective trophic effect on the retina and can survive long-term if not indefinitely after implantation into the vitreous.

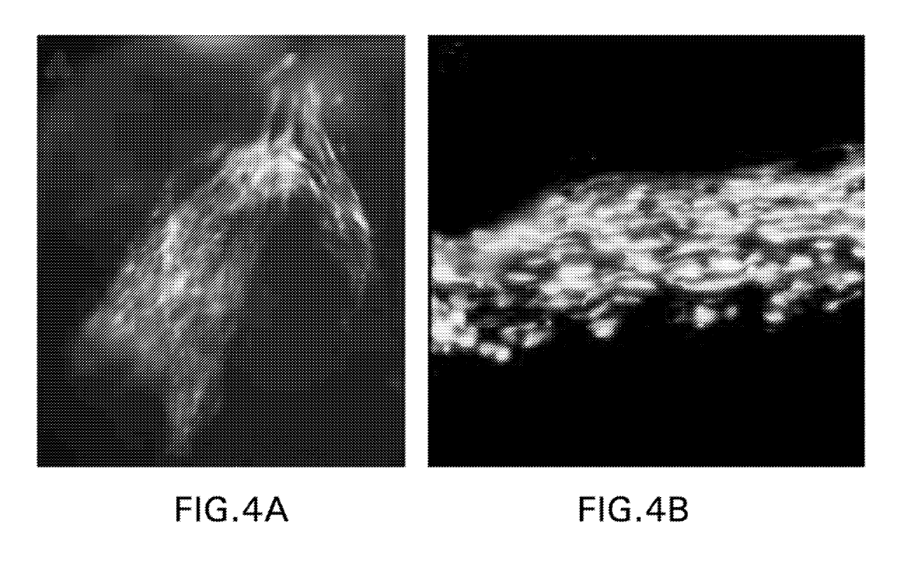

GFP-expressing bone marrow-derived mouse mesenchymal stem cells (MSCs) from normal mice were implanted into the vitreous of mice undergoing retinal degeneration due to a mutation the PPT1 gene. Unlike the neutralized embryonic stem cells, the implanted MSCs showed almost no integration into the host retina (FIG. 4). The donor cells formed a mesh-like sheet in the vitreous near the retinal surface but did not adhere to or invade the host retina (FIG. 4). These sheets of donor cells were stable in the vitreous for long periods of time and did not appear to change in size or location for up to 20 weeks after implantation. The presence of these cells in the vitreous for this long period of time did not have any apparent adverse effects on either the retina or the lens. These studies demonstrated that bone marrow derived MSCs implanted into the vitreous, because of their long-term survival and lack of toxic effects, were a potential vehicle for long-term delivery of therapeutic agents to the retina.

### Example 5

### Culture and Transduction of Canine MSCs

The mouse studies described in Example 4 demonstrated that MSCs can be safely implanted into the vitreous and survive for long periods of time (possibly indefinitely), indicating that transgenic autologous MSCs implanted into the vitreous could act as long-term sources of therapeutic agents for the retina. However, the substantial anatomic differences between human and mouse eyes raised the concern that the mouse findings may not be translatable to humans. Anatomically and physiologically, dog eyes and the dog CNS are much closer to those of humans than are mouse eyes and CNS. Therefore, because of the availability of the previously described canine CLN2 model, it was selected for assessment of whether implantation of genetically modified autologous MSCs can be used to treat retinal and CNS degenerative diseases, with the expectation that any findings in the dogs would more likely be translatable to humans in humans than experimental findings in mice.

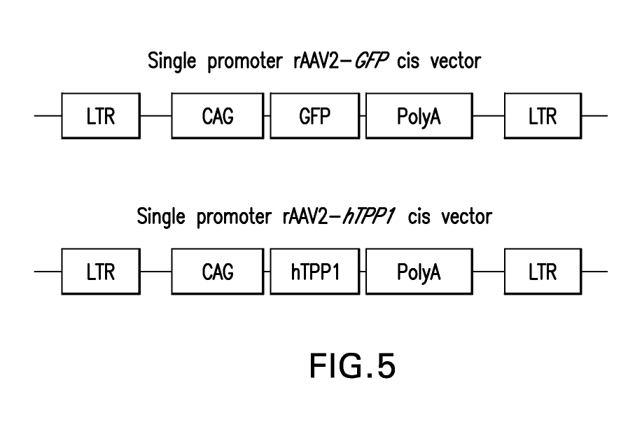

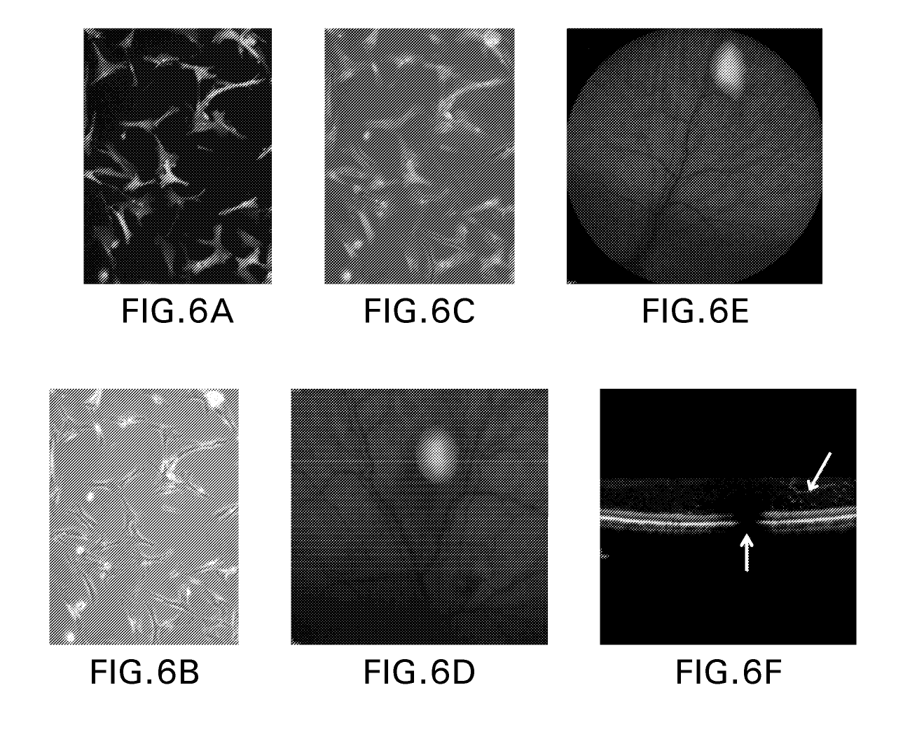

Bone marrow was aspirated from a 2-month-old dog that was homozygous for the TPP1 null allele. MSCs were grown from this aspirate using established techniques (Sanders, Autologous bone marrow-derived mesenchymal stem cell transplantation as a therapy for neuronal ceroid lipofuscinosis. In Pathobiology, pp. 167, University of Missouri, Columbia, 2007). The cells were transduced with a construct directing constitutive production of green fluorescent protein (GFP) using a recombinant adeno-associated virus serotype 2 (rAAV2-GFP) as a delivery vehicle (FIG. 5). Expression of the GFP transgene was monitored by UV fluorescence. The transduced cells expressed GFP in long-term culture without diminution of GFP fluorescence for at least 2 months (FIG. 6). These cells were implanted into the vitreous of a TPP1−/− dog and were monitored in vivo with autofluorescence imaging. The cells were found to survive long-term in the vitreous with no apparent diminution of the transgene expression (FIG. 6). A similar rAAV2 vector was produced with human pro-TPP1 cDNA (GenBank Accession No. NM_000391) in place of the GFP cDNA (rAAV2-CAG-TPP1) (FIG. 5).

### Example 6

### Transduction of Bone Marrow-Derived MSCs with a Construct Directing High Levels of Expression and Extracellular Release of TPP1 Enzyme

As discussed in detail above, high levels of long-lasting transgene expression in MSCs from dogs with CLN2 can be achieved after delivery of a DNA construct with rAAV2. A pro-TPP1 cDNA construct (rAAV2-CAG-TPP1) (FIG. 5) was delivered to MSCs derived from a TPP1−/− dog using the same rAAV2 vector under the same conditions as those used to transduce the cells with the GFP transgene. Success in transducing the MSCs was evaluated by measuring TPP1 enzyme activity in both the culture medium and the cultured cells. Enzyme activity was measured using an established method that employs a commercially available artificial substrate that is specific for TPP1 (Sohar et al., J. Neurochem. 73, 700-711, 1999).

For these studies, bone marrow aspirates were obtained under sterile conditions from the humerus bones of Dachshunds that were homozygous for the TPP1 null mutation. The samples were obtained when the dogs were 2 months of age. The aspirates were plated onto culture dishes and incubated at 37° C. in 5% CO₂ in MSC culture medium (Sanders, Autologous bone marrow-derived mesenchymal stem cell transplantation as a therapy for neuronal ceroid lipofuscinosis. In Pathobiology, pp. 167, University of Missouri, Columbia, 2007). The culture medium was changed daily until all of the nonadherent cells were removed. The adherent cells were grown to confluence and then split 1:1. At passage 3, after the cells reached confluence, the AAV2-TPP1 vector suspended in the culture medium was applied at a multiplicity of infection (MOI) of 50,000. The cells were incubated with the vector for 24 hours, after which the culture medium was replaced. Based on studies with the GFP vector, significant transgene expression was expected to be detectable at approximately 5 days after the transduction and to reach a plateau by 10 days post-transduction. To determine whether this was the case, aliquots of the culture medium and of the transduced cells were collected at 5, 10, 15, and 20 days after transduction and assayed for TPP1 enzyme activity levels. To assay release of TPP1 into the culture medium, for each time point, the medium was replaced 24 hours before sampling so that the amount of enzyme released could be determined. Immediately after sampling the medium, the cells were harvested and counted so that the TPP1 output per cell could be calculated. Some of the cells were homogenized and the intracellular TPP1 activity per cell were determined in the homogenates (Sohar et al., J. Neurochem. 73, 700-711, 1999). These analyses demonstrated that the transduced cells produced and released large amounts of TPP1 and enabled estimation of the amount of TPP1 that would be delivered to the vitreous per cell implanted.

### Example 7

### Implantation of Transgenic MSCs into the Vitreous of the Eyes from the Same Dogs from which Bone Marrow Cells were Derived

Transduced MSCs were implanted into the vitreous within one month of when the bone marrow aspirates were obtained, or when the dogs were approximately 3 months of age, prior to the onset of detectable retinal degeneration. Passage 3 cells were harvested and suspended in culture medium without serum. Cell density in the suspensions was determined with a hemocytometer. The cells were then concentrated by centrifugation and resuspended in the medium at a concentration to deliver the desired number of cells in an injection volume of 160 μl. A total of 500,000 cells were implanted into the vitreous of the first dog, and based on this outcome, the dose of MSCs implanted in subsequent dogs was adjusted as needed. Initially only one eye of each dog received the cell implants, while the other eye was sham-injected with medium alone to serve as a control. If any cross-correction from one eye to the other was seen, resulting from TPP1 getting into the bloodstream from the implanted eye and being transported to the contralateral eye, subsequent dogs received cell implants in both eyes and sham-treated dogs served as controls.

For implantation, the dogs were sedated and cells were injected into the vitreous just superior to the posterior pole. The globe was injected with a 27-gauge 1-inch sterile needle and syringe containing from 160 μl to 300 μl of the cell suspension or medium alone.

### Example 8

### Monitoring of Long-Term Survival of Donor Cells in the Eye, Delivery of TPP1 from these Cells to the Retina, and Ability of Cell Implants to Impede CLN2-Related Retinal Degeneration

OCT and SLO Imaging.

OCT imaging was used to monitor the survival and position of the implanted cells in the vitreous. Studies with mice indicated that the implanted cells form a mesh-like sheet near the retinal surface that can be visualized with OCT. The treated eyes were monitored with OCT at periodic intervals starting one week after implantation to assess the size and location of the sheet of implanted cells. OCT imaging was also employed to assess the efficacy of the implanted transgenic cells to prevent the disease-related retinal pathology. As described above, CLN2 in the Dachshund model was characterized by a dramatic thinning of the inner retinal layers and by localized retinal detachments (FIGS. 2 and 3). Using OCT, the ability of the cell implants to inhibit these pathological changes was evaluated. The localized retinal detachments were visualized over a large area of the retina using SLO (FIG. 3), and therefore, SLO was also employed to assess whether the implanted cells were able to prevent formation of the retinal detachments.

ERGs and PLRs.

As the disease progressed in dogs with CLN2, retinal degeneration was accompanied by progressive declines in ERG and PLR amplitudes (Whiting et al., Exp Eye Res 125:164-172, 2014). It was hypothesized that the functional impairments indicated by these changes would be inhibited by the presence of the TPP1-expressing cells implanted into the vitreous. To test this hypothesis, established protocols will be used to assess the ERG and PLR in the treated and sham-treated eyes at monthly intervals, starting just prior to the cell implantation. The functional impairments indicated by these changes were inhibited by the presence of the cells implanted into the vitreous.

### Example 9

### Analysis of Morphology for Retinal Cell Loss and Other Pathological Changes and Retinal Levels of TPP1 Activity

Morphology for Retinal Cell Loss and Other Pathological Changes.

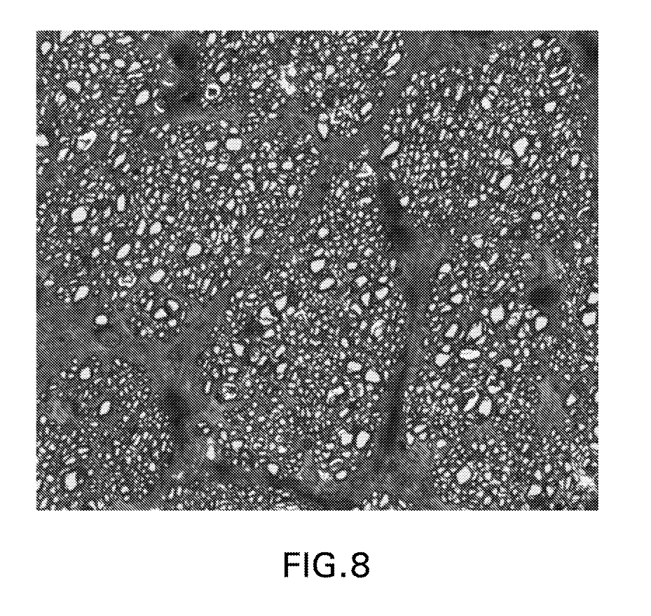

In the absence of TPP1 delivery to the CNS, progression of neurological signs in the affected dogs necessitates euthanasia at 10.5 to 11 months of age. All of the dogs are euthanized at 10.5 months of age and the retinas are either prepared for morphological analyses or for measurement of TPP1 enzyme activity. Established light and electron microscopy techniques are used to determine whether the characteristic CLN2 changes seen in the Dachshund are ameliorated by the implanted cells. To determine whether ganglion cell loss occurring in this disease is the cause of the observed thinning of the inner retina and whether implantation of TPP1-expressing MSCs into the vitreous can ameliorate such loss, a method for determining the total number of ganglion cells in the retina is determined by counting axons in the optic nerve (FIG. 8).

TPP1−/− dogs that receive intravitreal implants of the transgenic autologous MSCs and TPP1−/− and TPP1+/+ dogs that receive sham intravitreal injections are euthanized and the eyes enucleated, each with approximately 1 cm of optic nerve attached. For morphological analyses, the eye is immersed in an electron microscopy fixative (Katz et al., Invest. Ophthalmol. Vis. Sci. 49, 2686-2695, 2008) immediately after enucleation, and the cornea, iris, lens, and vitreous are removed. The tissue is incubated in the fixative with gentle agitation at room temperature for approximately 24 hours before further dissection. A segment of the optic nerve is then dissected for processing and analysis as described above. The total number of axons is compared between the 3 groups of dogs. These analyses indicate whether CLN2 is accompanied by ganglion cell loss and whether implantation of the transgenic MSCs protects against such loss. The retinas are analyzed for the other morphological changes that occur in this disease. In addition, the histology of the implanted cells is examined.

Retinal Levels of TPP1 Activity.

To test whether the TPP1 enzyme produced by the implanted cells is taken up by the retina and distributed to all layers of the retina, the retinas are isolated from treated and control eyes collected at necropsy and TPP1 enzyme activity measured in homogenates of the tissues. To localize TPP1 in the retinas, eyes are fixed with paraformaldehyde and immunohistochemistry used to label TPP1 protein using a commercially available antibody (Proteintech 12479-1-AP).

### Example 10

### Implantation of TPP1-Expressing MSCs Resulted in Long-Term Preservation of Retinal Function

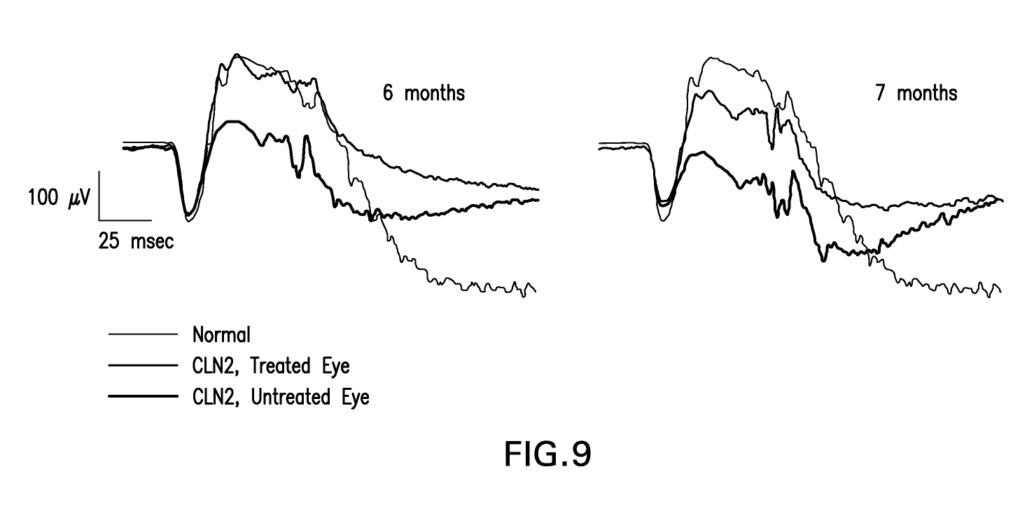

Two Dachshunds homozygous for the TPP1 null mutation, were used in a study to determine the efficacy of MSC-mediated delivery of the normal TPP1 protein to the retina in preventing loss of retinal function as assessed with ERG. Bone marrow was aspirated from a leg bone in each dog. MSCs from the marrow were grown in culture and then transduced with a TPP1 expression vector. After transduction, culture medium that the cells were growing in was collected and analyzed for TPP1 enzyme activity. This analysis confirmed that the cells were synthesizing and secreting functional TPP1 enzyme. At approximately 3.5 months of age, the transduced cells were implanted into the vitreous of the left eye (OS). The second dog was given 10 times the number of cells as the first dog, resulting in a much larger dose of TPP1 delivered to the retina. The right eye of both dogs (OD) was untreated. At 6 months of age (12 weeks after the cell injection), the ERG responses of both eyes were measured in each dog. Both dogs in the study showed a striking improvement in ERG b-wave amplitudes in the treated eye when compared to those of the untreated eye. The b-wave is the large positive deflection of the ERG waveform, which reflects activity of the second order neurons in the retina. In the untreated eye of both dogs, the b-wave was greatly reduced, indicating a loss of function of these second-level neurons. In the treated eyes, on the other hand, the b-wave was present at almost the same amplitude seen in normal dogs. FIG. 9 shows representative ERG tracings comparing the high-dose treated dog to a normal dog at 6 and 7 months of age.

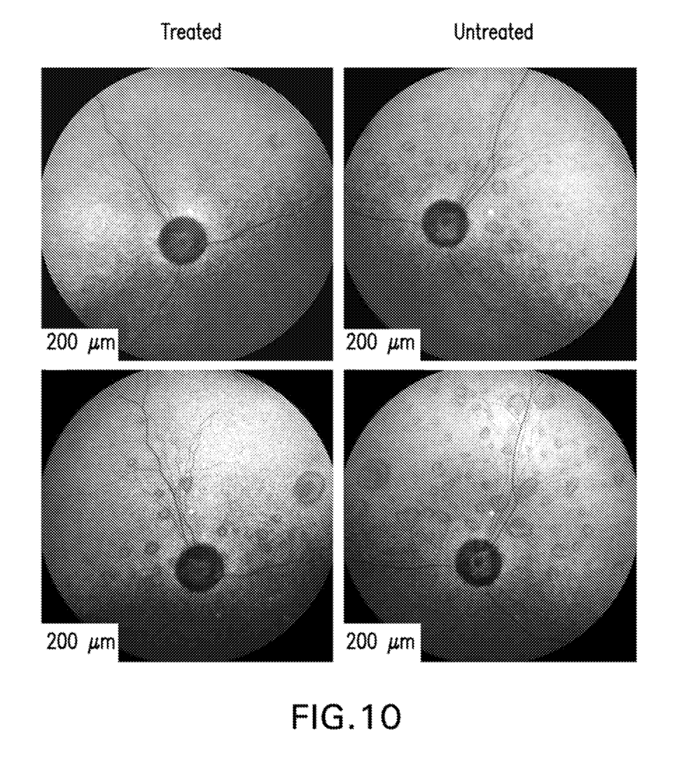

One of the treated dogs developed multifocal retinal detachments observed in most CLN2-affected dogs (FIG. 10). However, the prevalence of the lesions was much lower in the treated eye than in the untreated eye. These findings demonstrate that implantation of TPP1-expressing MSCs resulted in long-term preservation of retinal function, demonstrating the ability of this approach in preventing the progression of a retinal degenerative disease.

### Example 11

### Treatment of the Brain Using Transgenic MSCs Expressing TPP1

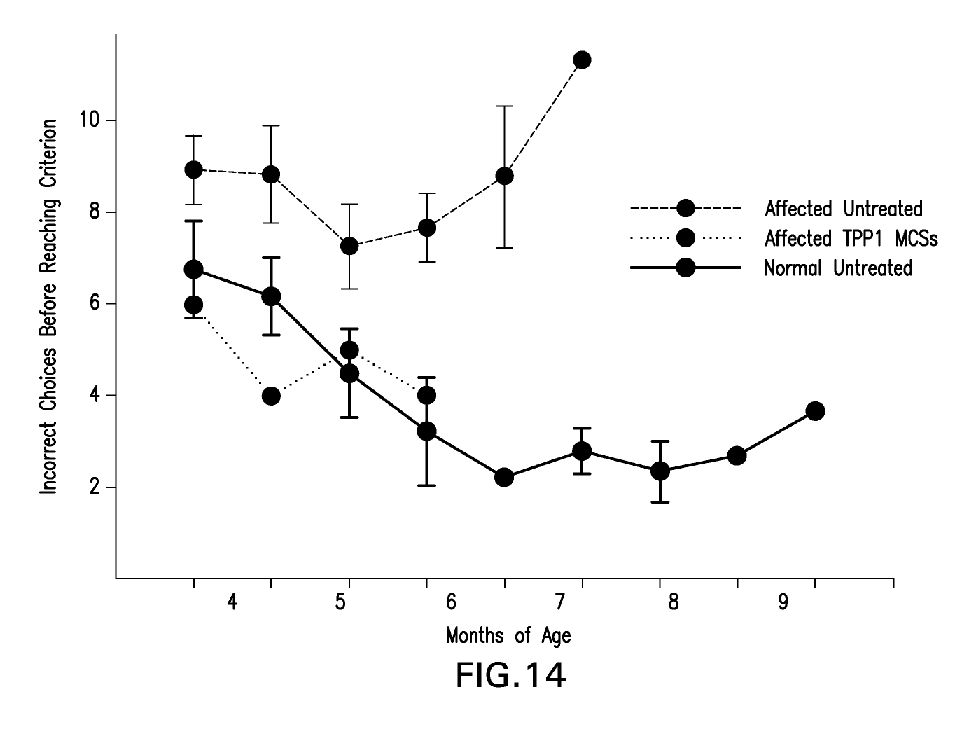

A preliminary study was conducted to test whether administration of TPP1-expressing MSCs to the CSF would have a therapeutic benefit in CNS function of TPP1−/− dogs. Untreated TPP1−/− dogs exhibit a severe impairment in cognitive function as measured by performance on a T-maze test (Sanders et al., Genes, Brain and Behavior 10: 798-804, 2011). As previously described, long-term administration of rhTPP1 to the CSF of affected dogs inhibits this disease-related impairment (Katz et al., J Neurosci Res 92:15911598, 2014). An experiment was conducted to determine whether administration of autologous TPP1-expressing MSCs to the CSF of a TPP1−/− dog would have a similar therapeutic benefit. It was found that a single administration of such cells early in the course of the disease resulted in a long-lasting improvement in T-maze performance compared to untreated affected dogs (FIG. 14). This preliminary study demonstrates that implanted MSCs producing the appropriate substance can have a therapeutic benefit not only for the retina but for the brain as well.

### Example 12

### Combined Treatment of the Retina and the Brain Using Transgenic MSCs Expressing TPP1

In order to treat both visual and neurologic symptoms of CLN2, both the retina and brain are treated using TPP1-producing stem cells, such as MSCs. To treat the retina, the cells are implanted into the vitreous of the eye, while the cells are implanted into the CSF to treat the brain. If treatment of the retina and brain are both effective, normalization of both retinal and CNS function should be seen.

Visual tests are developed for evaluating functional vision in the dogs used in the study based on a modification of the T-maze test. T-maze testing is typically performed to evaluate learning ability and cognitive function, however performance on the t-maze test is not affected by vision. The developed system is modified to rely on visual cues instead of learned patterns in order to evaluate vision in these dogs. Assessment of visually-mediated behavior is performed using a similar system developed for mice in order to give an objective measure of visual ability, as opposed to functional information about limited portions of the visual system. The system is designed with the ability to assess visual discrimination of light intensity, color, and pattern recognition.

What is claimed is:

1. A method of treatment or prevention of a neurodegenerative disease or retinal degenerative disease comprising delivery of at least one cell to a subject, wherein said cell provides a therapeutic compound to said subject in an effective amount to reduce disease symptoms.

2. The method of claim 1, wherein said cell comprises a construct expressing said therapeutic compound.

3. The method of claim 1, wherein delivery of the cell comprises injection into cerebrospinal fluid or the eye.

4. The method of claim 3, wherein injection into the cerebrospinal fluid comprises an intracerebroventricular, cisterna magna, or intrathecal injection, or a combination thereof.

5. The method of claim 3, wherein injection into the eye comprises injection into the vitreous.

6. The method of claim 1, wherein delivery of said at least one cell comprises injection into the cerebrospinal fluid and injection into the eye.

7. The method of claim 1, wherein said neurodegenerative disease is a central nervous system degenerative disease or a retinal degenerative disease.

8. The method of claim 7, wherein said retinal degenerative disease is a lysosomal storage disease or a neuronal ceroid lipofuscinosis disease.

9. The method of claim 1, wherein said neurodegenerative disease is caused by a mutation in the TPP1 gene.

10. The method of claim 1, wherein said neurodegenerative disease is characterized by progressive neurodegeneration, accumulation of autofluorescent lysosomal storage bodies, or blindness.

11. The method of claim 1, wherein said neurodegenerative disease is late-infantile neuronal ceroid lipofuscinosis or a disease homologous to late-infantile neuronal ceroid lipofuscinosis.

12. The method of claim 1, wherein said cell is an autologous cell.

13. The method of claim 12, wherein said autologous cell comprises a mesenchymal stem cell.

14. The method of claim 13, wherein said mesenchymal stem cell is isolated from bone marrow, adipose tissue, muscle tissue, blood, or dental pulp.

15. The method of claim 1, comprising delivery of a population of cells that provides a therapeutic compound to said subject in an effective amount to reduce disease symptoms.

16. The method of claim 1, wherein said therapeutic compound comprises a protein, a peptide, a polypeptide, an RNA molecule, a carbohydrate, an antibody or antibody fragment, or a small molecule that is not functionally present in cells of the subject.

17. The method of claim 16, wherein said therapeutic compound is a functional copy of a TPP1 protein.

18. The method of claim 17, wherein said TPP1 protein is a human TPP1 protein or a recombinant TPP1 protein.

19. The method of claim 1, wherein the subject is selected from the group consisting of murine, canine, feline, and human.

20. A construct for treating a neurodegenerative disease in a subject, the construct comprising a sequence expressing a therapeutic protein or peptide operably linked to a promoter that functions in an animal cell, wherein delivery of the construct to the subject in an effective amount reduces disease symptoms in the subject.

21. A cell comprising the construct of claim 20.

22. A composition for treating a neurodegenerative disease in a subject comprising at least one cell expressing a therapeutic protein or peptide in an effective amount to reduce disease symptoms and a carrier.

23. The composition of claim 22, wherein the cell is an autologous cell.

24. The composition of claim 23, wherein the autologous cell comprises a mesenchymal stem cell.

25. The composition of claim 24, wherein the mesenchymal stem cell is isolated from bone marrow, adipose tissue, muscle tissue, blood, or dental pulp.

26. The composition of claim 22, wherein the cell is delivered to the subject by injection into the cerebrospinal fluid or the eye.

27. The composition of claim 22, wherein the cell is delivered to the subject by injection into the cerebrospinal fluid and injection into the eye.

28. The method of claim 26, wherein injection into the cerebrospinal fluid comprises injection into the subarachnoid space or a ventricular space.

29. The composition of claim 26, wherein injection into the eye comprises injection into the vitreous.

30. The composition of claim 22, wherein the subject is selected from the group consisting of murine, canine, feline, and human.

31. A kit for treating a neurodegenerative disease in a subject comprising at least one cell from the subject expressing a therapeutic compound for treating a neurodegenerative disease in the subject.
